(19) United States
(12) Patent Application Publication
(10) Pub. No.: US 2016/0113561 A1
(43) Pub. Date: Apr. 28, 2016

(54) APPARATUS AND METHOD FOR DETECTING AND/OR MONITORING ONE OR MORE COMPOUNDS IN BLOOD

(71) Applicant: DAVID R. ELMALEH, NEWTON, MA (US)

(72) Inventor: DAVID R. ELMALEH, NEWTON, MA (US)

(21) Appl. No.: 14/963,195

(22) Filed: Dec. 8, 2015

Related U.S. Application Data
(63) Continuation-in-part of application No. 14/070,081, filed on Nov. 1, 2013.
(60) Provisional application No. 61/721,258, filed on Nov. 1, 2012.

(51) Int. Cl. A61B 5/15; A61B 5/145

(52) U.S. Cl. CPC A61B 5/150984; A61B 5/14532; A61B 5/150358

(57) ABSTRACT

A device for sampling blood comprising a test strip having a planar configuration having a top surface and a bottom surface, said bottom surface having one or more microneedles projecting therefrom and each microneedle having an elongated body having a first end and a second end, said first end forming a tip and said second end forming a base, said base affixed to said bottom surface of said platform.

### CROSS REFERENCE TO RELATED APPLICATIONS

This application is a continuation-in-part of U.S. patent application Ser. No. 14/070,081, filed Nov. 1, 2013, and claims the benefit of U.S. Provisional Patent Application No. 61/721,258, filed Nov. 1, 2012, each of which is hereby incorporated by reference in its entirety.

### FIELD OF THE INVENTION

Some embodiments of the present invention are directed to devices and methods for sampling blood, with particular application for the detection and monitoring of glucose for diabetic conditions.

### BACKGROUND OF THE INVENTION

Several conditions require sampling of blood. The sampling of blood can be used to determine the blood level of drugs with narrow therapeutic ranges, determine compliance or detect drugs of abuse. Blood sampling may also be necessary to determine adequate control of a disease state where, for example, without limitation, the patient can not regulate the level of a compound.

For example, there are two types of Diabetes Mellitus: type-1, which results from the body's failure to produce insulin; and type-2, which results from insulin resistance, a condition in which cells fail to use insulin properly, sometimes also with an absolute insulin deficiency. Globally, an estimated 387 million people have diabetes worldwide, with type-2 diabetes making up approximately 90% of the cases. This is equal to approximately 3.3% of the population, with equal rates in both women and men. In 2011, diabetes resulted in 1.4 million deaths worldwide, making it the 8^{th} leading cause of death. In 2014, this number rose to 4.9 million deaths. The number of people with diabetes is expected to rise to 592 million by 2035.

Diabetics do not have the ability to control glucose levels in the blood to maintain such levels in a normal range. Monitoring of glucose blood levels is needed in order to regulate the administration of insulin or other hypoglycemic or hyperglycemic agents.

Modern approaches to diabetes management include dietary and lifestyle management, often combined with regular ongoing blood glucose level monitoring. Optimal management of diabetes involves patients measuring and recording their own blood glucose levels. For patients on insulin, patient involvement is important in achieving effective dosing and timing, but requires significant commitment on the part of the patient.

Home blood glucose monitoring systems were developed to aid patient monitoring and have been around for several decades (see Clarke, S. F. and J. R. Foster, “A history of blood glucose meters and their role in self-monitoring of diabetes mellitus”, British Journal of Biomedical Science, 2012, 69(2), pgs. 83-93). Blood glucose meters/readers are medical devices for determining the approximate concentration of glucose in the blood. While most blood glucose readers today are small, portable electronic devices, blood glucose meters may also be a strip of glucose paper that may be dipped into a substance and measured to a glucose chart.

In modern blood glucose readers, a small drop of blood is obtained by a user by pricking the skin with a lancet and placing the drop on a disposable test strip that the meter reads to calculate the concentration of blood glucose of the user. In most instances, the size of the drop of blood needed by various readers varies from approximately 0.3 to 1 μl. Blood glucose levels are typically displayed by the reader in units of mg/dl or mmol/l.

Consumable and disposable test strips containing testing components that react with glucose in the drop of blood are typically used for each measurement with the reader. In certain systems, the test strip is a plastic test strip with a small spot impregnated with a testing substance, such as glucose oxidase and/or other testing substances. In most instances, each test strip is used once and then discarded, but other types of test strips are contemplated, such as, without limitation, reusable test strips permanently attached to a reader. For example, in some embodiments, readers use discs, drums, or cartridges that contain testing components for multiple uses.

Since test strips may vary from batch to batch, some models require the user to manually enter in a code found on a vial or container of test strips, or on a chip that comes with the test strip. By entering the coding or chip into the reader, the meter will be calibrated to that batch of test strips. Alternatively, some test strips may contain the code information in the strip. For example, a microchip may be included with a vial of test strips that can be inserted into the reader.

Innovations in the field of home blood glucose monitoring systems have significantly revolved around the testing components/reagents, the testing systems (e.g., electrochemical, reflectance photometry, etc.), the quantity of sample needed, the ease of use, and the addition of advanced microelectronics and software to perform a range of useful functions. In recent years, continuous glucose monitoring (CGM) systems have been developed to give greater insight into the direction, magnitude, duration, frequency and possible causes of glucose fluctuations in response to meals, insulin injections, hypoglycemic episodes and exercise throughout the day. Compared to conventional blood glucose measurements performed four to six times a day, results for the CGM systems are provided every 10 minutes for up to 72 hours.

Advancements in home blood concentration monitoring thus have focused on the device testing systems and ease of use, but not on the test strips. Despite these advancements, the need for the conventional “fingerprick” sample has not changed substantially, although recent advancements in lowering the quantity of sample needed for an accurate reading has led to the use of alternative sampling sites, such as the forearm or hand, which is intended to reduce sampling pain. Regardless, the need to stick the flesh with a lancet has remained, and patient compliance has historically suffered for it.

In a large multinational study, patients reported adherence rates for self-monitoring blood glucose systems of 70% and 64%, while diabetes healthcare providers' estimates were only 44% and 24% for Type-1 and Type-2 diabetes respectively (see, Clarke, S. F., referenced above). A number of barriers to optimal adherence to self-monitoring have been identified to include demographic factors and psychosocial elements (e.g., anxiety, self-perception of diabetes and vulnerability to complications), but also patient stress caused by the responsibility of self-care and the demands of regular, and possibly repeated, painful fingerpricks (Id.).

Despite this, testing strips used in conjunction with sampling meters (e.g., blood glucose monitoring systems) have largely remained unchanged, and steps to increase compliance have focused mainly on training and education (Id.). This is true despite the inconvenient need for the conventional “fingerprick” to obtain the blood sample. While certain advancements have allowed use of alternative sampling sites, which is intended to reduce sampling pain, blood concentration monitors still require piercing the flesh with a lancet.

Indeed, it has been suggested that there is a trend towards reduced investments in the development of new meter/strip technology, and there have been several failed attempts to develop alternative, notably non-invasive or minimally invasive glucose monitors (Id.). Accordingly, there is a long-felt, yet unresolved need to develop testing strips and/or monitoring systems that would aid in increasing patient compliance.

Accordingly, it would be desirable to be able to test blood levels of biologically important compounds, such as glucose, or drugs without discomfort or the aversion of sticking oneself with a lance.

### SUMMARY OF THE INVENTION

Human skin is a complex, multilayered tissue that serves the function of preventing water loss from the body and protecting the body against microorganisms and other exogenous materials by acting as a permeability barrier to the environment. The skin is the largest functional organ in the body, and its structure can be categorized into four main layers: the stratum corneum (SC), the viable epidermis (the SC and viable epidermis together make up the epidermis), the dermis, and the hypodermis (or subcutaneous fat layer).

The outermost superficial layer of the skin is called the stratum corneum and is between 10-20 μm thick. Below the stratum corneum lies the viable epidermis which has a thickness ranging between 50 and 100 μm. Directly below the epidermis lies a thicker region called the dermis having a thickness ranging between 1 and 2 mm. The dermis can be further sub-divided into two layers: the superficial papillary dermis and the reticular dermis. The papillary dermis is highly vascularized and consists of a rich bed of blood capillaries and lymphatic vessels which are found immediately below the epidermis. The overall vasculature nature of the dermis offers a rich blood flow of approximately 0.05 mL/min/mg, which is effective for the removal of molecules (including drugs) that have passed the outer layers of the skin, allowing for transport of drug molecules from the dermal-epidermal layer to the system circulation. The hypodermis lies beneath the dermis. It typically has a thickness of a few millimeters and also contains an extensive circulatory network as well as nerve fibers.

As known in the art, microneedles are similar to traditional needles, but fabricated on the micro scale. Microneedles are typically hundreds of microns long, 1 to 50 μm wide at the tip, and approximately 50-300 μm at the base. Microneedles may be fabricated from several different materials, such as, without limitation, metals, silicon, silicon dioxide, polymers, glass and other materials. They can be solid or hollow and can be fabricated as single needles or multi-needle arrays. Microneedles of various geometries and materials have been developed over the years, and, depending on the microneedle design, can be applied by several different delivery strategies for drug delivery.

As an alternative to the application of drug delivery, an alternative application of microneedles is the subject of the embodiments of the present invention. In particular, microneedles are typically long enough to reach the blood capillaries of the highly vascularized dermis, but too short to reach the nerve fibers found in the hypodermis. As such, microneedles have the capacity to draw small amounts of fluid through the skin without triggering a pain response from the nerve fibers, which makes them ideal for use with fluid (e.g. blood) sampling strips used in conjunction with fluid sampling devices such as, for example, blood glucose readers.

Some embodiments of the present invention feature devices and methods for sampling blood without pain and without initiating the psychological aversion of sticking oneself with a lance.

One embodiment, directed to a device for sampling blood, comprises a platform having a planar configuration having a top surface and a bottom surface. The bottom surface has one or more microneedles projecting therefrom and each microneedle has an elongated body having a first end and a second end. The first end is formed as a tip and the second end is formed as a base affixed to the bottom surface of the platform. The elongated body has a diameter of about 50 to 500 microns and a length of about 200 to 1500 microns. The platform has a passage opening in the top surface and a passage extending therefrom to a withdrawal opening in at least one of the bottom surface, the elongated body and at or about the tip. The withdrawal opening in the bottom surface in an area is closely associated with one or more microneedle bases for cooperating with one or more microneedles to receive a fluid sample withdrawn from the skin surface as the one or more microneedles are placed on and pressed into the skin of a user. The withdrawal opening in the elongated member or at or about the tip is for receiving one or more fluid samples withdrawn as the one or more microneedles are placed on and pressed into the skin of a user. The microneedles, having a small size in diameter and in length, do not reach nerve ending and do not elicit a pain response.

In the location in the elongated member or tip, the withdrawal opening is closer to blood vessels. The position of the withdrawal opening can be a mixture of locations and comprise all locations. For example, without limitation the microneedles may have a cross sectional “U” shape and define a withdrawal opening from the bottom surface to along the elongated member to the tip.

Some embodiments of the present device feature a platform having an array or plurality of microneedles. For example, one array features between 1-50 microneedles, and more preferably 1-10 microneedles.

Some embodiments of the present invention are ideally suited for use with a reader. The reader analyses the sample to determine the presence or absence or quantity of one or more compounds of interest. For example, without limitation, for individuals afflicted with diabetes, the compound of interest is glucose. Readers for monitoring glucose levels are well known in the art. Some embodiments of the present invention feature a platform of the present device affixed to the reader such that the passage is in fluid communication with the test elements of the reader. The device of the present invention can be permanently affixed or releaseably affixed allowing the devices to be removed after usage and replaced with unused devices. Replaceable devices allow the user to be more certain as to the clean and preferably sterile or near sterile microneedle surfaces.

Some readers feature capillary channeling strips for receiving a blood sample. Such channeling strips or test strips can be integrally affixed to the platform or be removably received on the platform. Some embodiments of the present invention feature devices for use with such readers. One embodiment features a device further comprising a platform cover forming a conduit in communication with the passage opening for transporting blood or a blood fraction to a reader. A further embodiment comprises a test strip received or integrally affixed to a least one of the platform and platform cover in fluid communication with the conduit for transporting blood or a blood fraction to the reader. Fluid moves through the test strip by capillary action, laminar flow, vacuum, adsorption or other means.

One platform cover of the present invention features window means which allows the user to see that the conduit has a blood sample. As used herein, window means refers to a transparent or semi transparent film, membrane, plastic piece which permits one to visualize the conduit from empty to having sample, by color change or direct visualization of the sample.

Some embodiments of the present invention feature a device further comprising packaging for holding said platform in a controlled environment. A controlled environment in the present context refers to clean, sterile or near sterile status. Some embodiments of the present invention feature packaging that facilitate use of the device. For example, one embodiment features a plurality of platforms aligned in strips or groups with each platform secured in a separate controlled environment. The groups comprise number of platforms which a user is likely to use during a day, or the strips allow the user to determine a number of platforms desired and allow such number to be separated from a bulk supply of platforms by a tear strip.

Some embodiments of the present invention are ideally suited for monitoring blood glucose levels. Diabetics need to check glucose levels regularly and drawing blood is one of the most unpleasant aspects of the disease.

A further embodiment of the present invention is directed to a method of sampling blood. The method comprises the step of providing a platform having a planar configuration, a top surface and a bottom surface. The bottom surface has one or more microneedles projecting therefrom and each microneedle has an elongated body having a first end and a second end. The first end is formed as a tip and the second end is formed as a base. The elongated body has a diameter of about 50-500 microns and a length of 200-1500-microns. The base is affixed to the bottom surface of the platform. The platform has a passage opening in the top surface and a passage extending therefrom to a withdrawal opening in at least one of the bottom surface, the elongated body and at or about the tip. The withdrawal opening in the bottom surface is in an area closely associated with one or more microneedle bases for cooperating with one or more microneedles to receive a fluid sample withdrawn from the skin surface as the one or more microneedles are placed on and pressed into the skin of a user. The withdrawal opening in the elongated member or at or about said tip for receiving one or more fluid samples withdrawn as the one or more microneedles are placed on and pressed into the skin of a user. The method further comprises the step of placing the platform on the skin of a user and pressing the microneedles into the skin to place the withdrawal opening in fluid communication with the user's blood and withdrawing a sample through the passage and out of the passage opening. The microneedles, having a small size in diameter and in length, have minimal or no interaction with nerve ending and do not elicit a pain response.

Some embodiments of the present method feature use of a reader to which the platform is attached, integrally affixed to or in fluid communication. For example, the method comprises the step of placing a platform on a reader or placing a platform in fluid communication with a reader and withdrawing a sample which is received and processed by the reader. One embodiment features window means to allow the additional step of checking the platform for the presence of sample.

Some embodiments of the present invention feature a device for sampling blood comprising a test strip having a planar configuration having a top surface and a bottom surface, the bottom surface having one or more microneedles projecting therefrom, each microneedle having an elongated body with a first end and a second end, the first end forming a tip and the second end forming a base, which base is affixed to the bottom surface of the platform. In certain embodiments, the one or more microneedles are arranged directly on a surface of the test strip.

Some embodiments feature a test strip having an array, or plurality, of microneedles embedded on the strip. The test strip may have a shape and configuration as generally known in the art with the addition of an array of microneedles embedded thereon. For example, in one embodiment, the strip may have a planar, elongated configuration having a top surface, a bottom surface, a proximal end and a distal end. The bottom surface of the test strip may have one or more microneedles projecting therefrom and each microneedle may have an elongated body having a first end and a second end. The first end may be formed as a tip and the second end may be formed as a base affixed to the bottom surface of the strip. The elongated body may have a diameter of about 50 to 500 microns and a length of about 200 to 1500 microns.

In certain embodiments, the microneedles are arranged on the distal end of the test strip, while the proximal end is configured for insertion into a reader, although other configurations are contemplated. In certain embodiments, the test strip includes a capillary channeling system, which is configured to transport/channel a fluid sample for the distal end to the proximal end, or vice versa. In this embodiment, when the microneedles puncture the skin, a fluid sample is withdrawn from the skin which moves by, for example, capillary action, laminar flow, vacuum, absorption or other means, from the distal end of the strip toward the proximal end. At the proximal end, the fluid sample is detected and sampled by a reader as known in the art.

Another embodiment of the present invention features a test strip for receiving a blood sample having a square, rectangular or toroidal shape or configuration with a top surface and a bottom surface and having an array, or plurality, of microneedles embedded on the bottom surface of the strip. In this embodiment, a fluid sample withdrawn by the microneedles has a smaller distance to travel in order to be read by a reader because the fluid sample does not travel from a distal end of the strip to a proximal end thereof. Rather, the fluid sample travels from the bottom surface of the strip, wherein it is withdrawn by the microneedles, to the top surface of the strip, where it pools and is offered for collection and sampling by a reader.

Additional embodiments of the present invention include packaging for holding the channeling strips in a protected and controlled environment as discussed above.

A further embodiment of the present invention is directed to a method of sampling blood using the test described above. The method includes providing a test strip having a planar configuration having a top surface and a bottom surface, the bottom surface having one or more microneedles projecting therefrom, each microneedle having an elongated body with a first end and a second end, the first end forming a tip and the second end forming a base, which base is affixed to the bottom surface of the platform. In certain embodiments, the fluid collecting strip may have a plurality of microneedles arranged directly thereon. A further step of the method may include placing the test strip on the skin of a user and pressing the microneedles into the user's skin. Another step of the present method may include receiving one or more fluid samples on the test strip as the fluid is withdrawn from the skin surface. In certain embodiments, the strip is configured to pool the fluid sample on a surface of the test strip opposing the surface having the microneedles such that the fluid sample may be collected and sampled by a reader as known in the art. In other embodiments, the fluid sample moves, by capillary action or other active or passive movement, from a distal end of the strip to a proximal end thereof that is configured for insertion into a reader wherein the fluid sample is detected and sampled as known in the art.

Additional embodiments of the present invention may include a system and/or a kit for sampling blood including a test strip having one or more microneedles, or an array/plurality of microneedles, disposed directly on a surface thereof, and a reader configured to accept the test strip.

Again, embodiments of the present invention are ideally suited for glucose monitoring in blood sample of diabetics.

These and other features and advantages will be apparent to those skilled in the art upon viewing the drawings which are briefly described next and reading the detailed description that follows.

### BRIEF DESCRIPTION OF THE DRAWINGS

FIG. 1 is a slightly elevated view of a top surface of a device having features of the present invention;

FIG. 2 is a slightly elevated view of the bottom surface of a device having features of the present invention;

FIG. 3A depicts a microneedle of the present invention;

FIG. 3B depicts a microneedle of the present invention;

FIG. 3C depicts a microneedle of the present invention;

FIGS. 4A and 4B depict a device having a reader;

FIG. 5 depicts a device having a reader;

FIG. 6 depicts a device having a capillary strip that connects to a reader;

FIG. 7 depicts a bottom view of a test strip according to certain aspects of the present invention;

FIG. 8A depicts a bottom view of a test strip according certain aspects of the present invention; and

FIG. 8B depicts a top view of a test strip according to the embodiment depicted in FIG. 8A.

### DETAILED DESCRIPTION OF THE INVENTION

Embodiments of the present invention will now be described in detail with respect to devices and methods for sampling blood to monitor glucose levels in blood samples of diabetic individuals. The methods and devices described are considered to be the best mode to make and use embodiments of the present invention. However, such embodiments of the present invention are subject to modification and alteration and the best mode contemplated may change over time. Therefore, the present description should be considered exemplary and not limiting.

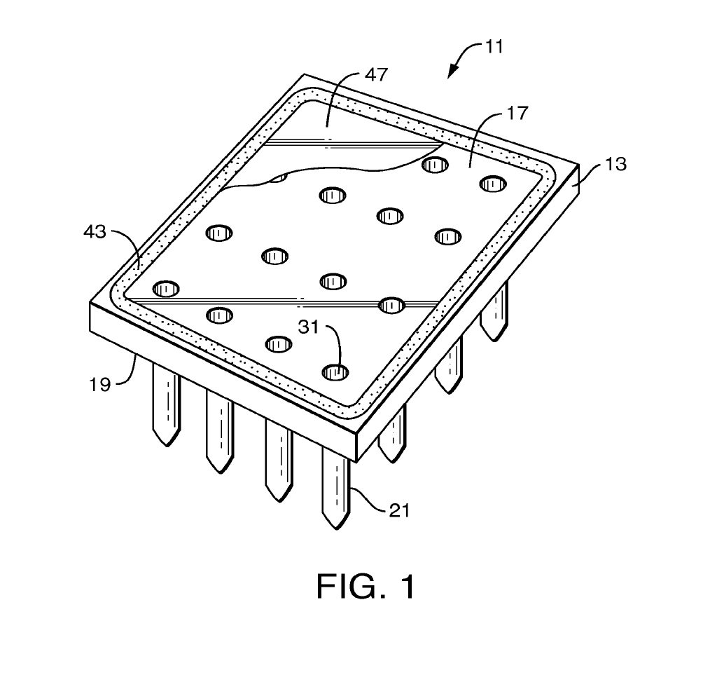

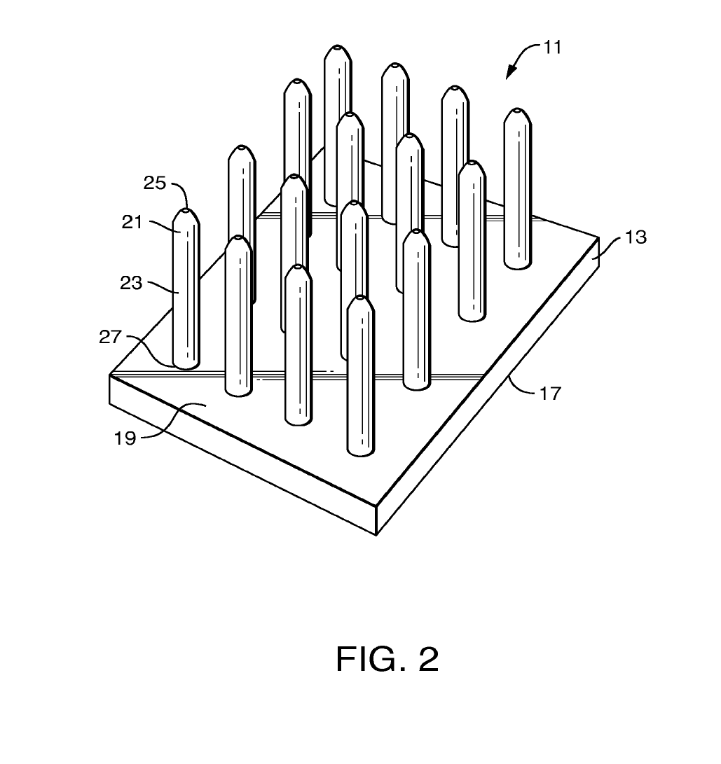

Turning now to FIG. 1, a device for sampling blood, generally designated by the numeral 11, is depicted. The device 11 has a platform 13 having a planar configuration, having a top surface 17 and a bottom surface 19, which is best seen in FIG. 2. Platform 13 is made of metals such as stainless steel, aluminum, plastics and the like.

Again, referring to FIG. 2, the bottom surface 19 has one or more microneedles, sixteen are depicted, which for the purpose of clarity only one is designated with the numeral 21. Each microneedle 21 projects from the bottom surface 19 and are arranged in an array. As used herein, the term “array” means a predetermined systematic arrangement of a plurality of microneedles. Although the array depicted is sixteen, smaller numbers of microneedles, as few as three or five, and as many as desired may used. However, to maintain the platform 13 in a size readily handled an upper limit of fifty to one hundred microneedles is preferred. Each side of platform 13 is approximately 0.2 to 0.5 cm.

Techniques for making platforms include those disclosed in Clinical Study: MTS (Microneedle Therapy System), 5-Month Study; Dr. K B Joon, Department of Dermatology, Dongguk University, South Korea (2006); Collagen Induction Therapy Comparison (IPL vs Micro-needling); Drs, H S Moon, S E Kim, D S Ko, A Y Lee, Dep. Of Dermatology, Eulji University School of Medicine and Dongguk University, S. Korea (2006); Product Comparison Study, MTS Roller vs Dermaroller); manufacturer's shelf-life and usage study, including known reference on materials and usage. Platforms can be purchased from 3M (Minneapolis, Minn., USA).

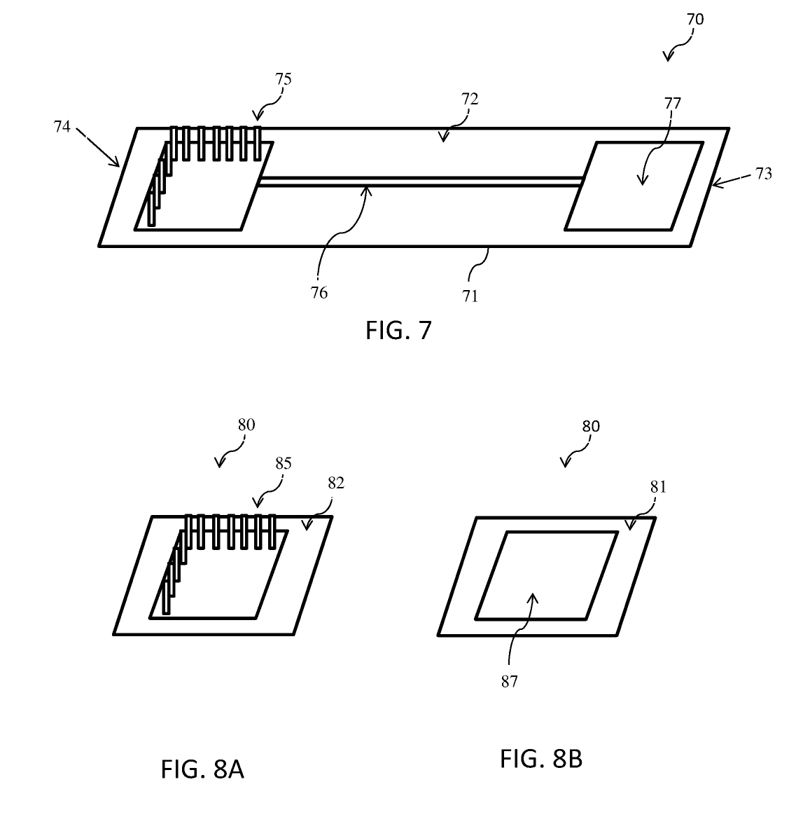

In other embodiments of the present invention, a platform may not be used, and microneedles may be arranged directly on a test strip, as discussed in more detail below. For example, platform 13 depicted in FIG. 2 may not be a separate platform upon which a test strip may be arranged, but may instead by an end of a test strip having a plurality of microneedles arranged thereon as discussed in more detail below with reference to FIG. 7.

Each microneedle 21 has an elongated body 23 having a first end 25 and a second end 27. The first end 25 is formed as a tip and this discussion will use the number designation 25 to refer to both the tip and the first end. The tip 25 is pointed to allow entry into the skin. The second end 27 is formed as a base affixed to the bottom surface 19 of the platform 13. This discussion will use the same number designation to refer to both the base and second end.

The elongated body 23 has a diameter of about 50 to 500 microns and a length of about 200 to 1500 microns. The length of the elongated body conforms to the normal thickness of the total epidermis layer of the skin. The majority of nerve endings are located deeper in the skin such that a microneedle 21, extending its full length into the skin, is unlikely to elicit a response from a nerve. Therefore, the microneedle 21 can be pressed into the skin without eliciting a pain response. The small diameter of each microneedle 21 minimizes the damage to skin layers.

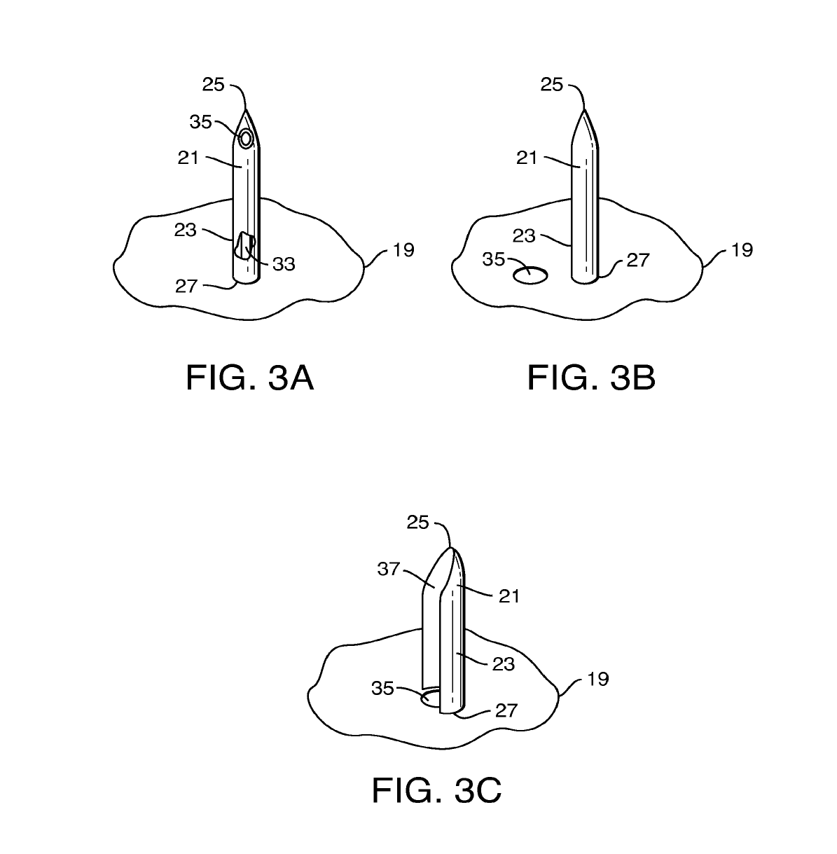

Returning now to FIG. 1, the platform 13 has a passage opening 31 in the top surface 17. Turning now to FIG. 3A, which depicts a single microneedle 21, the passage opening 31 is part of a passage 33 extends to a withdrawal opening 35 in the elongated body 23 at or about the tip 25. The withdrawal opening 35 in the elongated member 23 or at or about the tip 25 is for receiving one or more fluid samples withdrawn as the one or more microneedles are placed on and pressed into the skin of a user.

In the alternative, referring now to FIG. 3B, the passage 33 may be through the platform 13 with the withdrawal opening 35 in the bottom surface 19 in an area is closely associated with one or more microneedle bases 27. The withdrawal opening 35 cooperates with one or more microneedles 21 to receive a fluid sample withdrawn from the skin surface as the microneedle or microneedles 21 are placed on and pressed into the skin of a user.

FIG. 3C depicts a further alternative in which the passage 33 is through the platform 13 with the withdrawal opening 35 in the bottom surface 19 in an area closely associated with one microneedle base 27. The microneedle 21 is formed with a U shaped cross section having a hollow area 37 which functions as an extension of the passage 33 when the microneedle 21 is pressed into the skin of a user. The platform 13 may present a single form of microneedle 21 in an array or a mixture of forms of microneedles 21 of the types represented by FIGS. 3A, 3B and 3C, and modifications of such forms.

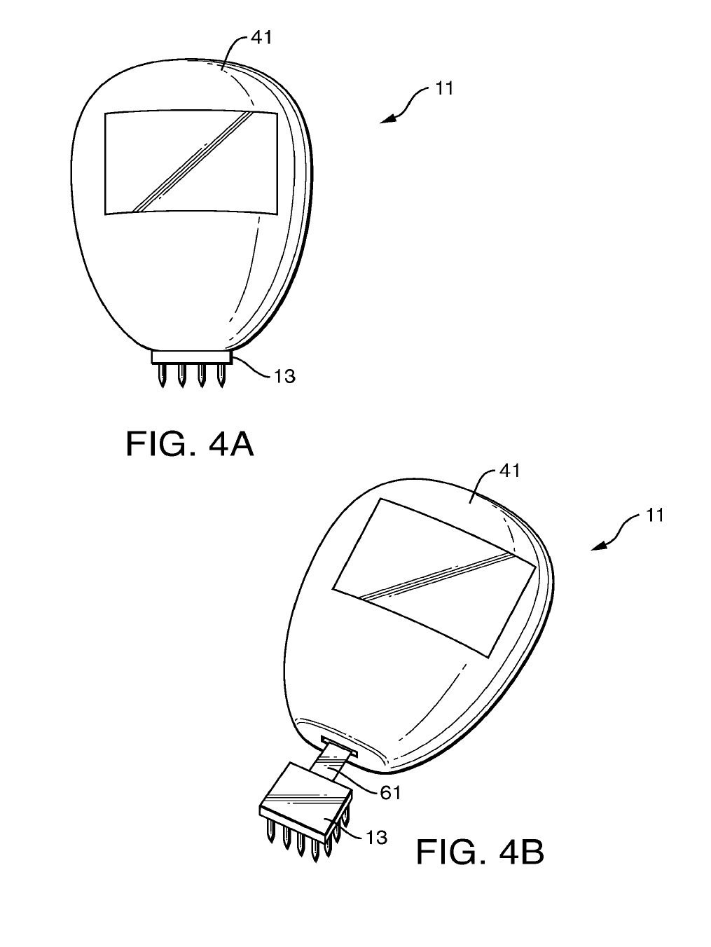

Turning now to FIGS. 4A and 4B, the platform 13 is suited for use with a reader 41. As used herein the term “reader” refers to an instrument that analyses the sample to determine the presence or absence or quantity of one or more compounds of interest. For example, without limitation, for individuals afflicted with diabetes, the compound of interest is glucose. Readers for monitoring glucose levels are well known in the art and are sold under several trademarks such as FREESTYLE FREEDOM®, FREESTYLE FLASH®, PRECISION XTRA®, (Abbott Diabetes Care Inc.), BREEZE 2®, CONTOUR® (Bayer), ONETOUCH®ULTRA® (Lifescan, Inc), ACCU-CHEK AVIVA®, ACCU-CHEK ADVANTAGE®, ACCU-CHEK COMPACT PLUS® (Roche), and others. These readers 41 typically have a screen for displaying results but, in the alternative, may also comprise transmission means, for example BLUETOOTH® wireless communication transmitters and receivers, to send results to further receiving equipment [not shown].

FIG. 4A depicts a device 11 having a platform 13 affixed to a reader 41 such that the passage 33 is in fluid communication with the test elements of the reader 41. The platform 13 can be permanently affixed or releaseably affixed to the reader 41. Platforms 13 that are releaseably affixed can be used and replaced with unused platforms 13. Returning now to FIG. 1, platform 13 has an adhesive strip 43 about the outer periphery of top surface 17 to adhere to a reader 41. In another alternative, the adhesive strip 43 coordinates with packaging to facilitate clean aseptic application of the device 11. Other means for affixing platform 13 to a reader 41 comprise mechanical clamping, interfitting or mating platform and reader parts and other means known in the art.

Many readers are intended to work with strips which filter or separate the components of the blood and pass the liquid fraction through one or more developing reagents such as glucose oxidase. Platform 13 receives such strips or filters, such as filter 47 (shown in partial cutaway so as to not obscure other features) in the area about the top surface 17. The filter 47 can be affixed to the platform 13 or placed on the top surface 17 by the user.

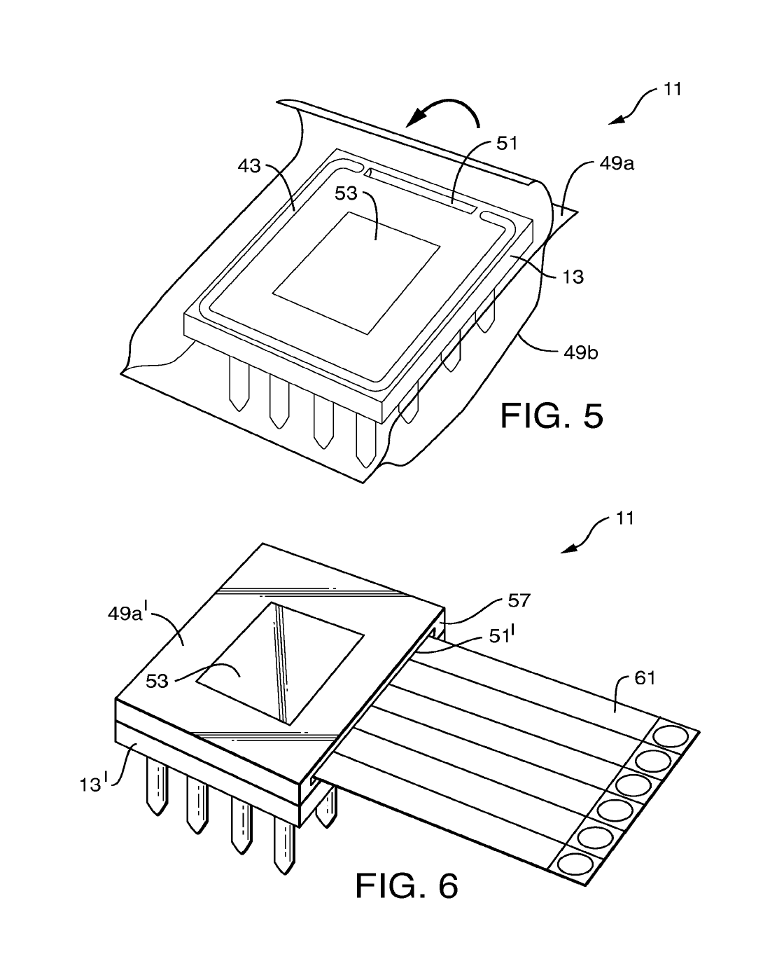

FIG. 4B depicts a device 11 having a reader 41 which receives test strip 61 of a conventional type in association with platform 13. FIG. 5 depicts a platform 13 having an adhesive strip 43 to which a cover sheet 49a is affixed. The device 11 has an open area 51 in which the cover sheet 49a is not secured to allow the open area to receive a test strip [not shown in FIG. 5] of the type known in the art and depicted with the device in FIG. 4B. In the alternative, the test strip is permanently affixed to the platform 13 with or without a cover sheet 49a to form a unitary structure which can be used and disposed of. The cover sheet 49a is used to facilitate handling the platform 13 as it is pressed in the skin.

FIG. 6 depicts a device 11 in which a rigid cover sheet 49a′ is affixed to platform 13′. Cover sheet 49a′ has a slot 57 creating an open area 51′ for receiving a test strip 61 which is permanently affixed to the platform 13′. The device 11 as depicted in FIG. 6 forms a unitary structure which can be used and then disposed of. Cover sheet 49a′ has a window 53′ or is comprised of a transparent or semi-transparent material to allow the user to see whether blood has been withdrawn and whether test strip 61 is properly in place. FIG. 4B depicts the platform 13′ of FIG. 6 with a test strip 61 and a reader 41.

As mentioned above, in certain, alternative embodiments of the present invention, a platform may not be used, and microneedles may be arranged directly on a test strip. For example, certain embodiments of the present invention feature a test strip for receiving a blood sample and having an array, or plurality, of microneedles embedded on the strip. The strip may have a shape and configuration as generally known in the art with the addition of an array of microneedles embedded thereon.

For example, in one embodiment depicted in FIG. 7, the test strip 70 may have a planar, elongated configuration having a top surface 71 (not seen in FIG. 7), a bottom surface 72 (shown face up in FIG. 7), a proximal end 73 and a distal end 74. The bottom surface 72 of the test strip 70 may have one or more microneedles 75 projecting therefrom, although the microneedles 75 may be arranged on any surface of the test strip 70. For the purposes of clarity, FIG. 7 depicts the first row and first column of an array of microneedles 75, but it is understood that additional rows and columns of microneedles 75 may be present on test strip 70.

Each microneedle 75 may have an elongated body having a first end and a second end as described above. The first end may be formed as a tip, and the second end may be formed as a base affixed to the test strip 70, e.g., at the bottom surface 72 thereof. In certain embodiments, microneedles 75 are arranged on the distal end 74 of the test strip as shown in FIG. 7, while the proximal end 73 is configured for insertion into a reader, although other configurations are contemplated.

In some embodiments, test strip 70 may include a capillary channeling system 76 for channeling fluid from distal end 74 to proximal end 73. Capillary channeling system 76 may consist of one or more capillaries or narrow tubes that extend along test strip 70 and are configured for channeling fluids therein. In certain embodiments, test strip 70 may have one or more apertures therethrough so that the fluid sample may travel from the skin and into the capillary system. In certain embodiments, the fluid sample soaks through test strip 70 into the capillary system?

In certain embodiments, bottom surface 72 of test strip 70 may include a detection area 77 at proximal end 73 thereof. Detection area 77 may include, for example, a component for detecting a substance within a fluid sample. In certain embodiments, detection area 77 may include glucose oxidase for detecting glucose in blood. Other detection components are contemplated.

In this embodiment, when the microneedles 75 puncture the skin, a fluid sample is withdrawn from the skin which moves by, for example, capillary action, laminar flow, vacuum, absorption or other means, through capillary channeling system 76 from the distal end 74 of test strip 70 toward the proximal end 73. At proximal end 73, the fluid sample is detected at detection area 77 and sampled by a reader as known in the art.

For example, with reference to FIG. 2, platform 13 may not be a separate platform upon which a test strip may be arranged, but may instead bee an end of a test strip having a plurality of microneedles arranged thereon. In this embodiment, device 11 may be a test strip (e.g., test strip 70), having a first end 13 including a bottom surface 19 and a top surface 17. Test strip 11 may be an elongated test strip as known in the art, and first end 13 may be an illustration of only a portion of test strip 11, the other portions of test strip 11 not being illustrated. First end 13 may include a plurality (or array) of microneedles 21 arranged on bottom surface 19 as shown. Microneedles 21 may be configured to puncture the skin of a user and withdraw an amount of fluid, for example, blood. Although not shown in FIG. 2, in certain embodiments first end 13 of test strip 11 may also include a capillary channeling system (e.g., capillary channeling system 76), as known in the art, which accepts fluid withdrawn by microneedles 21 and transports the fluid via capillary action to another portion of test strip 11.

In an alternative embodiment, FIGS. 8A and 8B illustrate the entirety of a test strip 80. In this embodiment, fluid withdrawn by microneedles 85 flows through test strip 80 from bottom surface 82 (shown face up in FIG. 8A) to top surface 81 (shown face up in FIG. 8B) where it pools on top surface 81 at pooling area 87 to be collected and/or read by a reader, such as reader 41. For the purposes of clarity, FIG. 7A depicts a first row and a first column of an array of microneedles 85, but it is understood that additional rows and columns of microneedles 85 may be present on test strip 80.

In some embodiments, a test strip 80 for receiving a fluid sample may have, without limitation, a square, rectangular or toroidal shape or configuration with a top surface 82 (shown face up in FIG. 8A), a bottom surface 81 (shown face up in FIG. 8B) and have an array, or plurality, of microneedles 85 embedded thereon, e.g., on bottom surface 82. Other shapes are contemplated, and embodiments of the present invention are not limited by the shape of testing strip 80. In this embodiment, a fluid sample withdrawn by the microneedles 85 has a smaller distance to travel to be read by a reader because the fluid sample does not travel from a distal end of the strip to a proximal end (as, for example, in FIG. 7). Rather, the fluid sample travels from the bottom surface 82 of the strip 80, wherein it is withdrawn by the microneedles 85, to the top surface 81 of the strip 80 where it pools at pooling area 87 and is offered for collection and sampling by a reader, such as, without limitation, reader 41.

In addition to FIG. 2, the latter embodiments regarding fluid test strips having microneedles embedded directly thereon may also be illustrated with reference to FIGS. 5 and 6.

With reference to FIG. 5 and the latter embodiments, platform 13 may, instead, by a test strip 13 having a top surface and a bottom surface, and having a plurality of microneedles arranged on the bottom surface as depicted in FIG. 5, similarly to the embodiment depicted in FIGS. 8A and 8B. In this particular embodiment, open area 51 for accepting a test strip is not needed and may not be present, and may include a pooling area 53, as shown. In this embodiment, the microneedles puncture the skin of a user and fluid flows from the skin through test strip 13 to pooling area 53.

With reference to FIG. 6 and the latter embodiments, cover sheet 49a′, slot 57, open area 51′ and window 53′ are not needed. Rather, a plurality of microneedles may be directly arranged on a bottom surface of a distal end of test strip 61, similarly to the embodiment depicted in FIG. 7. In this embodiment, test strip 61 may also include one or more capillary channeling systems (for example, the six channels depicted in FIG. 6, or capillary channeling system 76 in FIG. 7) for transporting fluid from a distal end to a proximal end of test strip 61 where it may collected and/or read by a reader, such as reader 41.

Embodiments of the present invention are ideally suited for monitoring blood glucose levels. Blood glucose monitoring reveals individual patterns of blood glucose changes, and helps in the planning of meals, activities, and at what time of day to take medications. Also, testing allows for quick response to high blood sugar (hyperglycemia) and low blood sugar (hypoglycemia), and might include diet adjustments, exercise and insulin. Diabetics need to check glucose levels regularly and drawing blood is one of the most unpleasant aspects of the disease.

Certain embodiments of the present invention feature a system and/or a kit including a blood glucose reader, such as reader 41, and at least one test strip having at least one microneedle disposed directly thereon. In this embodiment, the invention may feature a test strip (e.g., either a capillary channeling test strip 70 (see FIG. 7) or a pooling test strip 80 (see FIGS. 8A and 8B)) having a top surface, a bottom surface and at least one microneedle embedded directly on the bottom surface. In preferred embodiments, the test strip has a plurality, or array, of microneedles embedded thereon. In certain embodiments, the test strip may have an elongated shape with a proximal end and a distal end, wherein the microneedles are arranged on the distal end of the test strip as depicted, for example, in FIG. 7. In preferred embodiments, the test strip is configured for use directly with the reader. For example, a user may apply a test strip by pressing the microneedles embedded on the test strip into the skin thereby allowing fluid (e.g., blood) to be withdrawn from the skin. The fluid sample makes contact with the test strip and either travels the length of the test strip via capillary action, or pools on the surface of the strip opposing the microneedles. The reader then reads the test strip directly and calculates a concentration of a substance within the fluid sample. For example, a glucose concentrate in blood may be calculated.

The operation of the device 11 will now be described in relation to a method of sampling blood. The method comprises the step of providing a platform 13 having a planar configuration with a top surface 17 and a bottom surface 19.

The bottom surface 19 has one or more microneedles 21 projecting therefrom and each microneedle 21 has an elongated body 23 having a first end 25 and a second end 27. The first end 25 is formed as a tip and the second end 27 is formed as a base. The elongated body 23 has a diameter of about 50 to 500 microns and a length of 200 to 1500 microns. The base 27 is affixed to the bottom surface 19 of the platform 13. The platform 13 has a passage opening 31 in the top surface 19 and a passage 33 extending therefrom to a withdrawal opening 35 in at least one of the bottom surface 19, the elongated body 23 and at or about the tip 25.

The method further comprises the step of placing the platform 13 on the skin of a user and pressing the microneedles into the skin to place the withdrawal opening 35 in fluid communication with the user's blood and withdrawing a sample through the passage 33 and out of the passage opening 31.

The blood sample can thus be received directly into a reader 41 or by means of a test strip 61 which can be received by the platform 13 or integrally affixed thereto to form a unitary structure. The microneedles, having a small size in diameter and in length, do not reach nerve ending and do not illicit a pain response.

A further embodiment of the present invention is directed to a method of sampling blood using the test strips having microneedles embedded directly thereon as described above. The method includes providing a fluid collecting test strip having a plurality of microneedles arranged directly thereon (see, e.g., FIGS. 7, 8A and 8B), pressing the plurality of microneedles into a skin surface of a user, and receiving a fluid sample on the strip as the fluid is withdrawn from the skin surface. In certain embodiments, the strip is configured to pool the fluid sample an a surface of the strip opposing the microneedles such that the fluid sample may be collected and sampled by a reader as known in the art (see, for example, FIGS. 8A and 8B). In other embodiments, the fluid sample moves, by capillary action, or other active or passive movement, from a distal end of the strip to a proximal end that is configured for insertion into a reader wherein the fluid sample is detected and sampled as known in the art (see, for example, FIG. 7).

Thus, the description has presented the best mode contemplated for making and using the present invention with the understanding that the present invention is subject to modification and alteration without departing from the teaching herein. Therefore, the present invention should not be limited to the precise descriptions herein but should encompass the subject matter of the claims that follow and their equivalents.

1. A device for sampling blood comprising:
a test strip having a planar configuration having a top surface and a bottom surface,
said bottom surface having one or more microneedles projecting therefrom, each microneedle having an elongated body having a first end and a second end, said first end forming a tip and said second end forming a base, said base affixed to said bottom surface.

2. The device of claim 1 wherein said microneedles have a cross sectional shape and said shape is a “U”.

3. The device of claim 1 wherein said platform has an array of microneedles.

4. The device of claim 1 wherein said array has between 1 to 50 microneedles.

5. The device of claim 1 further comprising a distal end and a proximal end, wherein the microneedles are arranged on the distal end.

6. The device of claim 1, wherein the test strip further comprises a capillary channeling system.

7. The device of claim 5 further comprising a reader configured to accept the proximal end of the test strip.

8. The device of claim 7, wherein said reader detects levels of glucose in the blood or blood fraction.

9. A system for sampling blood comprising:
a test strip comprising a plurality of microneedles disposed directly on a surface thereof; and
a reader configured to accept the test strip.

10. The system of claim 9, wherein the test strip comprises a proximal end and a distal end, wherein the microneedles are disposed directly on a surface of the distal end, and the reader is configured to accept the proximal end of the test strip.

11. The system of claim 10, wherein the test strip further comprises a capillary channeling system for channeling a blood sample from the distal end to proximal end.

12. A method of sampling blood comprising the steps of:
providing a test strip having a planar configuration having a top surface and a bottom surface, said bottom surface having one or more microneedles projecting therefrom, each microneedle having an elongated body having a first end and a second end, said first end forming a tip and said second end forming a base, said base affixed to said bottom surface
placing said test strip on the skin of a user and pressing said microneedles into said skin; and
receiving one or more fluid samples withdrawn from the user into said test strip as a result of said one or more microneedles being placed on and pressed into the skin of a user.

13. The method of claim 12, wherein said test strip has an array of microneedles.

14. The method of claim 13, wherein said array has between 1 and 50 microneedles.

15. The method of claim 12, further comprising a capillary channeling system for transporting blood or a blood fraction to a reader.

16. The method of claim 12, wherein said one or more samples is received by a reader.

17. The method of claim 15, wherein said reader detects levels of glucose in the blood or blood fraction.
